(19) United States
(12) Patent Application Publication
(10) Pub. No.: US 2003/0191188 A1
(43) Pub. Date: Oct. 9, 2003

(54) Method for treating migraine symptoms with ibuprofen and salts thereof

(76) Inventor: Joseph R. Codispoti, Philadelphia, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/412,861

(22) Filed: Apr. 14, 2003

Related U.S. Application Data
(62) Division of application No. 09/709,069, filed on Nov. 9, 2000.
Application No. 09/709,069 is a continuation-in-part of application No. 09/449,124, filed on Nov. 24, 1999, now abandoned.

(51) Int. Cl. A61K 31/192

(52) U.S. Cl. 514/570

(57) ABSTRACT

The present invention provides a method for treating the photophobia and phonophobia symptoms of migraine attacks with an effective amount of ibuprofen, pharmaceutically acceptable salts thereof, isomers thereof, or mixtures thereof.

### CROSS-REFERENCE TO RELATED APPLICATION

This Application is a continuation-in-part application of U.S. application Ser. No. 09/449,124 filed on Nov. 24, 1999, which is incorporated herein by reference in its entirety.

### FIELD OF THE INVENTION

The present invention relates to the treatment of migraine symptoms by the administration of an effective amount of ibuprofen, more specifically the present invention relates to the treatment of phonophobia and photophobia with ibuprofen.

### BACKGROUND OF THE INVENTION

Migraine provides a wide variation of pain and symptoms, and associated disorders with those who suffer from the disease. Included among these symptoms are nausea, headache, moderate to severe pain, as well as incapacitating pain and total disability in a percentage of patients. Some patients also recite more specific symptoms such as photophobia, painful sensitivity to light; or phonophobia, painful sensitivity to sound.

Treatment of migraine pain includes both prescription and non-prescription or over-the-counter medication. Among the over-counter medication that is currently available is a combination of acetaminophen/aspirin/caffeine, as set forth in U.S. Pat. No. 5,972,916, the contents hereby incorporated by reference as set forth herein in its entirety. The combination of active ingredients suffers from the drawback that some of the actives might cause undesired side effects. For example, it is well know that aspirin can cause stomach irritation and caffeine can cause anxiety and sleeplessness.

Therefore there is a continuing need for treatment of migraine, as well as treatment alternatives to specific migraine symptoms.

### SUMMARY OF THE INVENTION

The present invention provides a method for treating photophobia and phonophobia associated with a migraine attack by providing an effective amount of ibuprofen, pharmaceutically acceptable salts thereof, isomers thereof, or mixtures thereof. The present invention relies on the action of a single active ingredient, i.e., ibuprofen, pharmaceutically acceptable salts thereof, isomers thereof, or mixtures thereof. The present invention also contemplates the use of additional ingredients, such as flavors, binders and excipients.

### DESCRIPTION OF THE DRAWINGS

FIG. 1 depicts the percentage of subjects with reduced photophobia over time after treatment with ibuprofen.

FIG. 2 depicts the percentage of subjects with reduced phonophobia over time over time after treatment with ibuprofen.

### DETAILED DESCRIPTION OF THE INVENTION

Ibuprofen is a well known analgesic material that has analgesic and antipyrritic properties. It is commercially available in various forms for many years.

According to the present invention, ibuprofen, pharmaceutically acceptable salts thereof, isomers thereof, or mixtures thereof, are preferably administered as an oral dose, as an effective dose to alleviate the photophobia and phonophobia symptoms, which comprises from about 100 to about 800 milligrams of ibuprofen per dose; preferably from 200 to about 600 and most preferably from about 300 to about 400 milligrams of ibuprofen per dose. Typical doses of the pharmaceutically acceptable salts of ibuprofen will vary according to the molecular weight of the salt required to give the equivalent dose of ibuprofen. Typical oral doses of pharmaceutically acceptable salts of ibuprofen range from about 110 to about 1700 milligrams per dose. Typical doeses of mixtures of ibuprofen or its isomers with pharmaceutically acceptable salts thereof range from about 100 to about 1700 milligrams per dose, preferably from about 200 to about 1300 milligrams per dose, and most preferably from about 300 to about 850 milligrams per dose. Typically doses are taken every four to six hours, but care should be taken to avoid exceeding the daily maximum recommended dosage of ibuprofen of 2400 milligrams per day.

Examples of suitable pharmaceutically acceptable salts of ibuprofen include any of the inorganic cation salts such as sodium, potassium, lithium, magnesium, calcium, cesium, ammonia, ferrous, zinc, manganous, aluminum, ferric, and manganic; organic salts of ibuprofen with primary, secondary, tertiary and quaternary amines, or mixtures thereof. Examples of such primary, secondary, tertiary and quaternary amines include substituted amines including but not limited to naturally occurring substituted amines, cyclic amines, basic ion exchange resins, and mixtures thereof. More specifically, suitable amines include but are not limited to triethylamine, tripropylamine, 2-dimethylaminoethanol, 2-diethylaminoethanol, lysine, arginine, histidine, caffeine, procain, N-ethylpiperidine, hydrabamine, choline, betaine, ethylenediamine, glucosamine, TRIS(hydroxymethyl)aminomethane, methylglycamine, theobromine, pruines, piperazine, piperidine, polyamine resins and the like, and mixtures thereof. In one embodiment, a mixture of ibuprofen and its sodium salt may be used.

Examples of suitable isomers include, but are not limited to R-ibuprofen, S-ibuprofen and mixtures thereof.

Suitable dosage forms include solids or liquids. Solid forms include tablets, capsules, liquid-filled soft gelatin capsules, powders, sachets and the like. Suitable liquids include suspensions, solutions, emulsions and the like.

The ibuprofen, pharmaceutically acceptable salts thereof, isomers thereof, or mixtures thereof, may be admixed with various pharmaceutically-acceptable excipients and other ingredients including but not limited to diluents, granulating agents, disintegrating agents, binding agents, lubricants and the like. Examples of these various ingredients are set forth in U.S. Pat. No. 5,660,860, the contents hereby incorporated by reference as if set forth in its entirety.

The present invention as set forth in the following examples are meant to exemplify the various aspects of carrying out the present invention and are not intended to limit the invention in any way.

### EXAMPLE 1

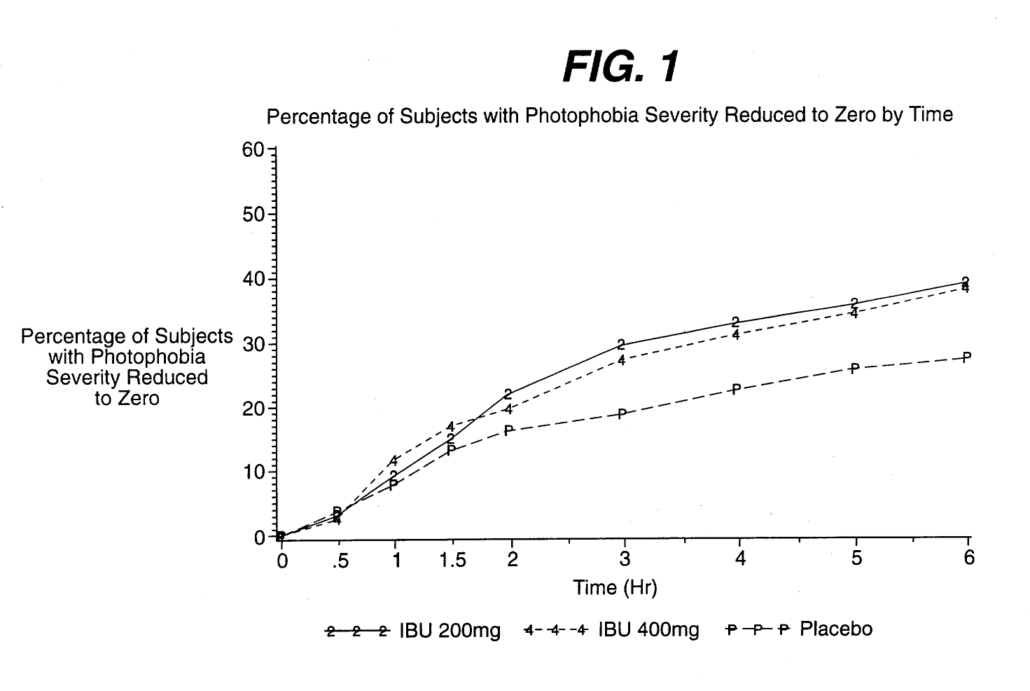

Subjects suffering from photophobia due to a migraine were either given a placebo or treated with 200 or 400 milligrams of ibuprofen. Approximately 650 subjects were in the study. Some subjects were given placebos, while others were given either 200 or 400 milligrams of ibuprofen. Periodic assessments of their photophobia were made and the results are presented in FIG. 1. The results indicate that at time of two hours or longer the treatment of photophobia with ibuprofen was effective in relieving photophobia

### EXAMPLE 2

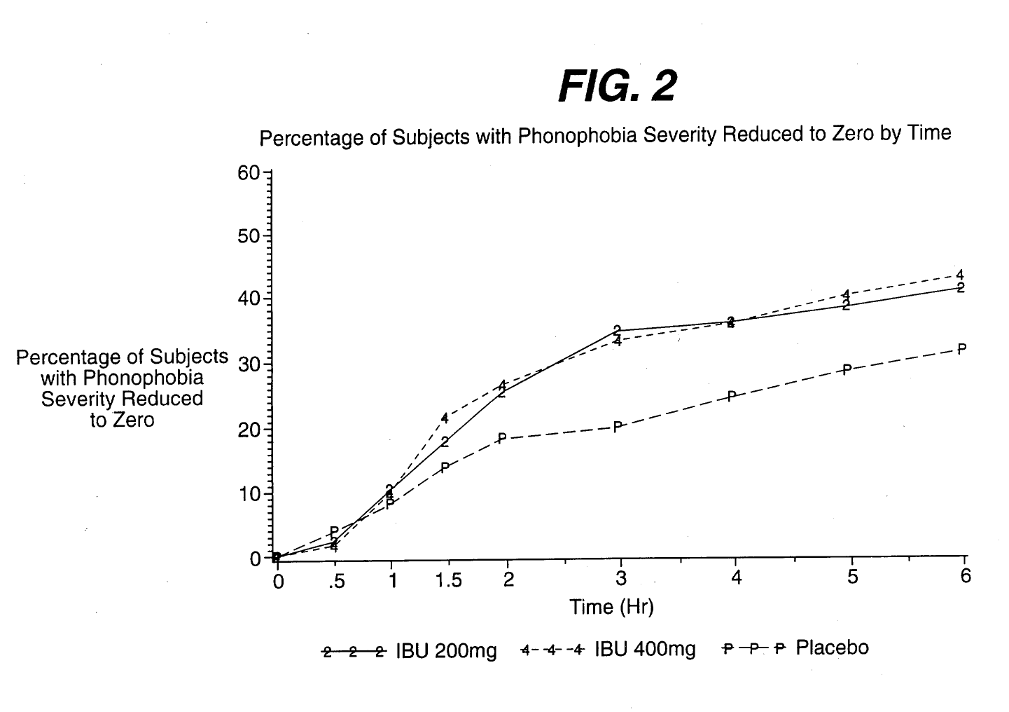

Subjects suffering from phonophobia due to a migraine were either given a placebo or treated with 200 or 400 milligrams of ibuprofen in tablet form. Periodic assessments of their photophobia were made and the results are presented in FIG. 2. The results indicate that at time of two hours or longer the treatment of photophobia with ibuprofen was effective in relieving photophobia.

I claim:

1. A method for mitigating or treating photophobia associated with migraine to a patient in need thereof comprising:
providing an effective amount of ibuprofen, pharmaceutically acceptable salts thereof, isomers thereof, or mixtures thereof, as the sole pharmaceutically effective ingredients.

2. The method of claim 1 wherein the amount of ibuprofen, isomers thereof, or mixtures thereof, is from about 100 to about 800 milligrams per dosage.

3. The method of claim 1 wherein the amount of ibuprofen, isomers thereof, or mixtures thereof, is from about 200 to about 600 milligrams per dose.

4. A method for mitigating or treating phonophobia associated with migraine to a patient in need thereof comprising:
providing an effective amount of ibuprofen, pharmaceutically acceptable salts thereof, isomers thereof, or mixtures thereof, as the sole pharmaceutically active ingredients.

5. The method of claim 4 wherein the amount of ibuprofen, isomers thereof, or mixtures thereof, is from about 100 to about 800 milligrams per dosage.

6. The method of claim 4 wherein the amount of ibuprofen, isomers thereof, or mixtures thereof, is from about 200 to about 600 milligrams per dose.

7. The method of claim 1 wherein the amount of ibuprofen, pharmaceutically acceptable salts thereof, isomers thereof, or mixtures thereof, is about 200 milligrams per dose.

8. The method of claim 1 wherein the amount of ibuprofen, pharmaceutically acceptable salts thereof, isomers thereof, or mixtures thereof, is about 400 milligrams per dose.

9. The method of claim 4 wherein the amount of ibuprofen, pharmaceutically acceptable salts thereof, isomers thereof, or mixtures thereof, is about 200 milligrams per dose.

10. The method of claim 4 wherein the amount of ibuprofen, pharmaceutically acceptable salts thereof, isomers thereof, or mixtures thereof, is about 400 milligrams per dose.

11. The method of claim 1 wherein the pharmaceutically acceptable salts of ibuprofen are selected from the group consisting of:
a) inorganic cation salts;
b) organic salts of ibuprofen with pharmaceutically acceptable primary, secondary, tertiary, and quaternary amines; and
c) mixtures thereof.

12. The method of claim 1 wherein the pharmaceutically acceptable salt of ibuprofen is:
a) an inorganic cation salt selected from sodium, potassium, lithium, magnesium, calcium, cesium, ammonia, ferrous, zinc, manganous, aluminum, ferric, and manganic;
b) an organic salt of ibuprofen with primary, secondary, tertiary and quaternary amines selected from triethylamine, tripropylamine, 2-dimethylaminoethanol, 2-diethylaminoethanol, lysine, arginine, histidine, caffeine, procain, N-ethylpiperidine, hydrabamine, choline, betaine, ethylenediamine, glucosamine, TRIS(hydroxymethyl)aminomethane, methylglycamine, theobromine, pruines, piperazine, piperidine, and polyamine resins; or
c) mixtures thereof.

13. The method of claim 1 wherein the mixture is a mixture of ibuprofen and its potassium salt.

14. The method of claim 1 wherein the isomer of ibuprofen is selected from the group consisting of R-ibuprofen, S-ibuprofen and mixtures thereof.

15. The method of claim 4 wherein the pharmaceutically acceptable salts of ibuprofen are selected from the group consisting of:
a) inorganic cation salts;
b) organic salts of ibuprofen with pharmaceutically acceptable primary, secondary, tertiary, and quaternary amines; and
c) mixtures thereof.

16. The method of claim 4 wherein the pharmaceutically acceptable salt of ibuprofen is:
a) an inorganic cation salt selected from sodium, potassium, lithium, magnesium, calcium, cesium, ammonia, ferrous, zinc, manganous, aluminum, ferric, and manganic;
b) an organic salt of ibuprofen with primary, secondary, tertiary and quaternary amines selected from triethylamine, tripropylamine, 2-dimethylaminoethanol, 2-diethylaminoethanol, lysine, arginine, histidine, caffeine, procain, N-ethylpiperidine, hydrabamine, choline, betaine, ethylenediamine, glucosamine, TRIS(hydroxymethyl)aminomethane, methylglycamine, theobromine, pruines, piperazine, piperidine, and polyamine resins; or
c) mixtures thereof.

17. The method of claim 4 wherein the mixture is a mixture of ibuprofen and its potassium salt.

18. The method of claim 4 wherein the isomer of ibuprofen is selected from the group consisting of R-ibuprofen, S-ibuprofen and mixtures thereof.

19. The method of claim 1 wherein the amount of the pharmaceutically acceptable salts of ibuprofen is from about 100 to about 1700 milligrams per dose.

20. The method of claim 4 wherein the amount of the pharmaceutically acceptable salts of ibuprofen is from about 100 to about 1700 milligrams per dose.

21. The method of claim 1 wherein the amount of the mixtures of ibuprofen, isomeres thereof, and pharmaceutically acceptable salts thereof is from about 100 to about 1700 milligrams per dose.

22. The method of claim 1 wherein the amount of the mixtures of ibuprofen, isomeres thereof, and pharmaceutically acceptable salts thereof is from about 200 to about 1300 milligrams per dose.

23. The method of claim 4 wherein the amount of the mixtures of ibuprofen, isomers thereof, and pharmaceutically acceptable salts thereof is from about 100 to about 1700 milligrams per dose.

24. The method of claim 4 wherein the amount of the mixtures of ibuprofen, isomers thereof, and pharmaceutically acceptable salts thereof is from about 200 to about 1300 milligrams per dose.

25. A method for mitigating or treating photophobia associated with migraine to a patient in need thereof comprising:
providing an effective amount of ibuprofen as the sole pharmaceutically effective ingredient.

26. A method for mitigating or treating phonophobia associated with migraine to a patient in need thereof comprising:
providing an effective amount of ibuprofen as the sole pharmaceutically active ingredient.
